(19) United States
(12) Patent Application Publication
(10) Pub. No.: US 2011/0245902 A1
(43) Pub. Date: Oct. 6, 2011

(54) METHOD FOR RELIEVING MOTION SICKNESS AND RELATED APPARATUS

(76) Inventor: Jay W. Katz, Delray Beach, FL (US)

(21) Appl. No.: 12/750,358

(22) Filed: Mar. 30, 2010

(51) Int. Cl. A61F 7/12

(52) U.S. Cl. 607/113

(57) ABSTRACT

The invention is directed toward a non-chemical method of relieving motion sickness that first includes the step of refrigerating an enhanced earplug. The enhanced earplug has a first portion having a handle and connector. The second portion having a first end and a corresponding second end where the first end is affixed to the first portion through the connector. This second portion also includes a deformable bladder having a cavity and a quantity of coolant maintained within the deformable bladder. The second step is to insert the enhanced earplug into a user's ear canal through use of the handle. The third step is to cool the vestibular system of the user placing the coolant close to the ear drum. A fourth step includes removing the enhanced earplug. These four steps are repeated as necessary prior to engaging in an event, which could cause motion sickness.

### FIELD OF THE INVENTION

This invention is directed toward a method of relieving motion sickness through a two-part constructed enhanced earplug that includes a deformable bladder having a cavity filled with a coolant for placement proximate to the vestibular system. More specifically, the method is directed to repeated placement of the coolant within the ear canal to create adaptation.

### BACKGROUND OF THE INVENTION

Motion sickness, also known as kinetosis, is a medical condition caused by a disagreement between visually perceived movement and the vestibular system's sense of movement. Put another way, motion sickness is generally caused by the difference between how an individual's eyes perceive movement in comparison to how the inner ear senses the same stimuli. Motion sickness, depending upon the underlying cause, is also commonly referred to as seasickness, car sickness, air sickness or more generally stimulation sickness.

Studies have shown that over ⅓rd of the population is susceptible to some form of motion sickness even in mild circumstances—such as being on a boat in calm water. These same studies find that over ⅔rds of the population are susceptible to the condition in even more severe conditions. Individuals suffering from motion sickness will exhibit a variety of symptoms including dizziness, fatigue and nausea. Yet another similar condition, called “sophite syndrome” can occur where the individual exhibits tiredness. If the motion sickness becomes prolonged, the individual may vomit. However, such vomiting tends not to relieve the condition.

There are three primary categories of treatment for the condition of motion sickness: chemical, electrical and natural. Chemical treatment options include medications such as Dramamine or Bonine/Antivert. Transdermal patches that include a quantity of scopolamine (1.5 mg) have also been used as a treatment option. General pharmacological treatments for nausea and vomiting can be employed to treat minor conditions. Sedating anti-histamines, such as promethazine, also improve minor motion sickness, although they do cause side affects such as drowsiness. While many of the medical options provide relief from motion sickness, they do require ingestion of chemicals and possible side effects—which deter many individuals from seeking treatment.

NASA has performed extensive research on the use of electric forms of treatment for motion sickness. Over half of astronauts have reporting suffering some form of motion sickness while in space flight. The most severe reports of such “space sickness” come during an astronaut's maiden flight. One treatment option developed by NASA for those suffering from motion sickness is a form of LCD shutter glasses to create a stroboscopic vision of 4 hz with a dwell of 10 milliseconds. This system is generally taught and disclosed in United States Pat. No. 6,932,090.

There exist only rudamentary non-chemical and non-electrical forms of treatment for motion sickness. These natural treatments include fixating on a stationary position while in a moving vehicle. One example includes looking out of the window of a moving vehicle to gaze toward the horizon—which helps reorient the inner sense of balance. Having the individual close his or her eyes is another common natural treatment option. Napping or sleeping can also help off-set the physical condition of motion sickness. Chewing gum sometimes improves the condition—specifically when dealing with car sickness. However, none of the aforementioned natural treatment options provides complete or lasting relief. Moreover, none of these natural options helps provide a permanent solution to severe conditions.

Accordingly, there is a need in the art of treating motion sickness for a non-chemical and non-electrical method. Such device should be portable, reusable and non-toxic. More importantly, there is a need for a simple yet effective device to provide a non-chemical form of treatment for individuals adverse to taking prescribed or over-the-counter medications. Lastly, the method of treatment should be robust and provide long-term relief even for severe conditions.

### SUMMARY OF THE INVENTION

This invention offers a non-chemical and non-electrical form of relieving and decreasing sensitivity to all types of motion sickness that is portable, reusable and non-toxic. Moreover, the invention helps treat symptoms of motion sickness caused by both short-term and prolonged linear movement, such as a cruise, airplane flight or similar experience. The invention is based upon a two-part constructed enhanced earplug having a first portion and corresponding second portion.

The first portion includes an engaging rod that maintains and secures the components of the first portion, which include a handle, diaphragm and connector. The second portion includes a deformable bladder, which has an opening and a cylindrical sheath that forms a cavity. The opening is secured to the connector located on the first portion. A quantity of coolant (which may be in the form of a gel) is maintained within the cavity. A sufficient quantity of coolant should be used in order to sufficiently cool the vestibular system—when the enhanced earplug is placed within a user's ear canal proximate to the tympanic membrane.

The invention is further directed to a method of using the enhanced earplug to relieve symptoms of motion sickness. Preferably, each ear canal is pre-treated through rinsing with a cooled liquid, which can be water. The method begins with refrigerating the enhanced earplug in order to chill the coolant housed within the deformable bladder. Next, the method contemplates inserting the enhanced earplug into a user's ear canal through use of the handle. Thus, the deformable bladder is positioned proximate to the tympanic membrane. The third step is to cool the vestibular system of the user through the proximity of the coolant to the various otolith organs. Cooling the fluid within these otolith organs and the related semi-circular canals decreases sensitivity of the ciliary bundles of hair cells when interacting with octoconia crystals during linear movement, which reduces potential confusion with visually perceived movement. After a prescribed period of time, the enhanced earplug is removed from the ear canal.

These four steps can be repeated, as necessary, prior to engaging in any event which could potentially cause symptoms associated with motion sickness. Preferably, the four steps are repeated each of the three days prior to the implicated event. Moreover, the method contemplates performing a single treatment of the four steps each day during a prolonged event which risks motion sickness—such as a cruise. Such protocol improves the chance of adaptation of the vestibular system to become less sensitive and accordingly reduce any potential disagreement with visually perceived movement.

### BRIEF DESCRIPTION OF THE DRAWINGS

For a fuller understanding of the invention, reference is made to the following detailed description, taken in connection with the accompanying drawings illustrating various embodiments of the present invention, in which:

FIG. 1 illustrates both the various components of outer, middle and inner ear as well as the enhanced earplug;

FIG. 2 is side view of the enhanced earplug including the deformable bladder filled with a coolant; and

FIG. 3 is a front view of the enhanced earplug showing the first portion and corresponding second portion.

### DETAILED DESCRIPTION OF EMBODIMENTS OF THE INVENTION

The present invention will now be described more fully hereinafter with reference to the accompanying drawings, in which embodiments of the invention are shown by way of example. This invention may be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

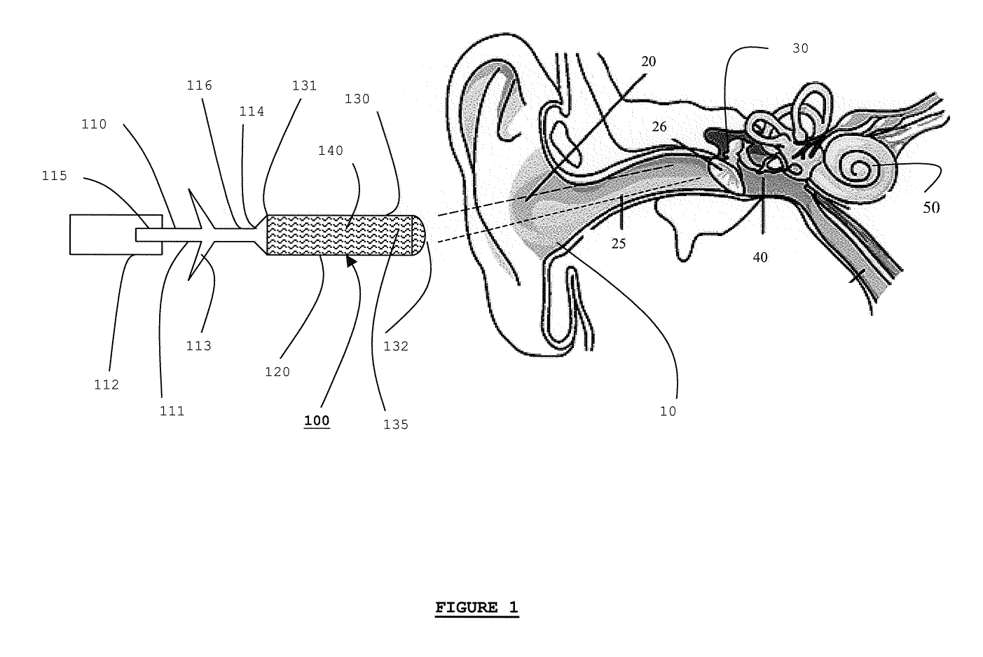

FIG. 1 illustrates both the primary components of the human ear 10 as well as one apparatus contemplated by the invention. As shown in FIG. 1, there are three primary components of the human ear 10: the outer ear 20, middle ear 30 and inner ear 40. The outer ear 20 extends from outside of the body, through the ear canal 25 and ends at the tympanic membrane 26 (more commonly known as the ear drum). As further illustrated, the middle ear 30 includes an air filled cavity filled with three tiny bones—referred to as ossicles—which transmit and amplify sound between the tympanic membrane 26 and the cochlea. While the middle ear 30 is primarily an air filled cavity, the inner ear 40 is composed of the fluid filled cochlea and the vestibular system 50.

The vestibular system 50 functions to provide an individual's sense of balance, motion and position (in combination with the eyes and the propioceptors in the feet). It is comprised of three semi-circular canals connected to two membranes called the saccule and utricle. Both membranes are referred to as the otolith organs. Both organs help sense direction and speed, including both linear and angular acceleration of the body. Within the otolith organs are multiple layers including a viscous gel layer and the otoconia layer. The otolith organs are filled with an endolymphatic fluid, which includes otoconia crystals. These crystals are located in the otoconia layer and are heavier than their surroundings. When linear acceleration occurs, these crystals deflect ciliary bundles of hair cells and produce a sensory signal called tricular signals.

Most utricular signals elicit eye movements. Interpretation of otolith signals by the brain are often difficult—especially when it comes to odd linear movements. When these otolith signals begin to differ from visual perception, the result is the dizziness and nausea commonly classified as motion sickness. It is the focus of the present invention that direct treatment of the vestibular system 50 to reduce conflict with visual perception represents a non-chemical and effective way to reduce motion sickness.

### The Overall Apparatus

FIGS. 1 through 3 offer, by way of example, one apparatus contemplated by the invention to treat the vestibular system 50. Other related embodiments will be known and understood by those of ordinary skill in the art now having the benefit of the teachings of the present invention. First turning to FIG. 1, the apparatus is directed to a two-piece constructed enhanced earplug 100. The primary function of this enhanced earplug 100 is to deliver a sufficient quantity of coolant proximate to the tympanic membrane 26 located at the end of the ear canal 25 (which is enhanced by fluid instilled and trapped within the tympanic membrane 26).

As further shown in FIG. 1, the enhanced earplug 100 includes a first portion 110 and a corresponding second portion 120. The first portion 110 functions to navigate the enhanced earplug 100 into the ear canal 25, as well as effectuate a seal with the outer ear 20. The second portion 120 provides the requisite treatment to decrease sensitivity of the vestibular system 50 and thus relieve symptoms common to motion sickness.

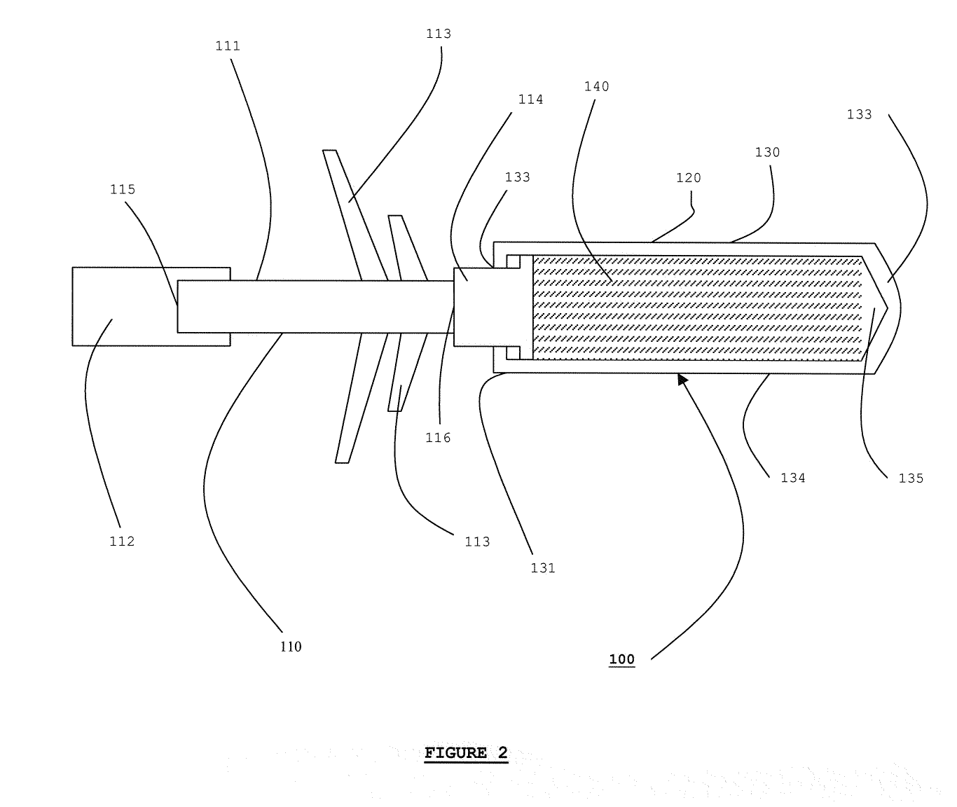

FIG. 2 offers a more detailed illustration of the various components of both the first portion 110 and second portion 120 of the enhanced earplug 100. As shown, the first portion 100 may include an engaging rod 111, a handle 112, one or more diaphragms 113 and a connector 114. The engaging rod 111 includes a first side 115 and a corresponding second side 116. The engaging rod 111 is positioned throughout the first portion 110 and connects all of the various components 112-114 together.

As shown in both FIG. 1 and FIG. 2, the handle 112 is positioned proximate to the first side 115 of the engaging rod 111. Preferably, the handle 112 is essentially cylindrical in shape and is of a sufficient size and dimension to be handled by a user's thumb and index fingers to navigate the enhanced earplug 100 for positioning within the ear canal 25. Optionally, the handle 112 can be made of a soft, spongy and pliable material such as a foam or neoprene. However, the handle 112 should nonetheless provide sufficient stability and rigidity.

Positioned proximate to the handle 112 are the one or more diaphragms 113. The diaphragms 113 act as dampers to sufficiently engage and create a seal between the engaging rod 111 and the outer opening of the outer ear 20. The diaphragms 113 are preferably made out of a soft, resilient and deformable material such as rubber, silicone, polymer or similar material known to those of ordinary skill in the art. The connector 114 connects the second end 116 of the engaging rod 111 to the second portion 120.

As further shown in FIG. 2, the second portion 120 includes a deformable bladder 130 filled with a coolant gel 140. It is the coolant gel 140, positioned proximate to the tympanic membrane 26 (shown in FIG. 1), that ultimately treats the vestibular system 50 to relieve and/or reduce symptoms associated motion sickness. The deformable bladder 130 includes a first end 131 and a corresponding second end 132 (shown in FIG. 2). The first end 131 includes an opening 133 of sufficient size and dimension to engage, fit over and/or create an effective seal with the connector 114 of the second portion 120. Positioned between both ends 131 and 132 is a cylindrical sheath 134. The cylindrical sheath 134 includes cavity 135 of sufficient size and dimension to house and store a sufficient quantity of the coolant gel 140. Put another way, the cavity 135 includes an internal volume that allows coolant gel 140 to be maintained within the deformable bladder 130.

The coolant gel 140 illustrated in both FIG. 1 and FIG. 2 is preferably made of a non-toxic and commercially available refrigerant. Alternatively, the coolant gel 140 can be any substance capable of cooling the inner ear 40—including, but certainly not limited to, any form of liquid or even a non-toxic metal sheath. Such coolant gel 140 should be selected to provide an efficient level and period of treatment, through ultimately cooling the various parts of the vestibular system 50, including the otolith organs. Preferably, the coolant gel 140 should be made of a water soluble cellulose ether, which can be a food grade gel.

The deformable bladder 130 and quantity of coolant gel 140 can be scaled according to the unique shape, volume and size of an individual's ear canal. For example an adult may require a larger deformable bladder 130 compared to a child. Likewise, a more severe condition may require a large amount of coolant gel 140. Again, the key design consideration is to have a sufficient amount of coolant gel 140 to indirectly cool the inner ear 40, including the endolymphatic fluid and octoconia crystals/sensitive vestibular nerves located in the octoconia layer of these organs and nerves.

By cooling these various portions of the vestibular system 50, the sensitivity of the various hairs within these organs and nerves is temporarily decreased. Accordingly, there exists less of a risk of confusion between the vestibular system 50 and visual perception, which reduces the risk and/or symptoms associated with motion sickness.

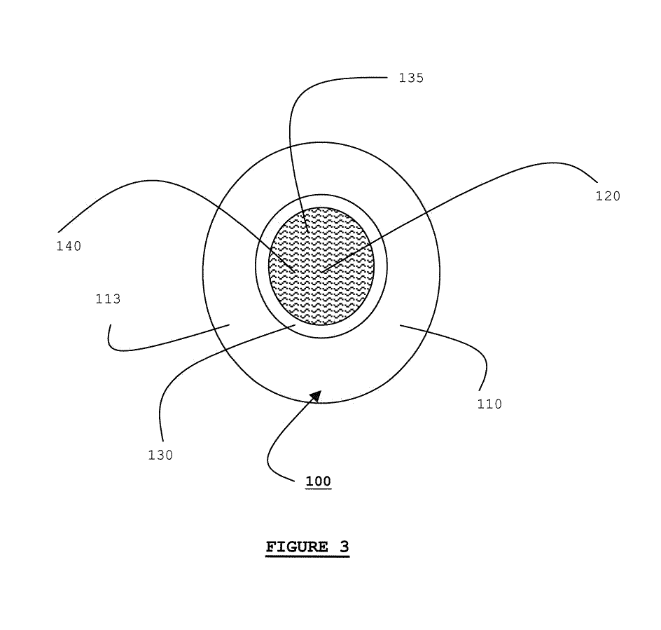

FIG. 3 offers a front view of the enhanced earplug 100, showing the different diameters of the first portion 110 and second portion 120.

### Method of Use

In addition to the aforementioned apparatus, the invention is further directed to a method of relieving and decreasing sensitivity to motion sickness. As has been described, the primary function of the enhanced earplug 100 is to deliver a sufficient amount of coolant—maintained within the second portion 120 of the apparatus—proximate to the tympanic membrane 26 to cool (and therefore reduce sensitivity of) the vestibular system 50. Prior to beginning the main method of treatment, it is preferably to rinse the ear canal 25 with a cooling bath. The cooling bath can be water or any non-toxic liquid. This treatment helps begin the process of cooling the components of the inner ear prior to the first step discusses in detail below.

The initial step of the method is to refrigerate at least the second portion 120 of the enhanced earplug 100. This can be accomplished through placing it in a refrigerator, an ice bath or similar cooling system. Preferably, the second portion 120 should be cooled between 40 to 60 degrees Fahrenheit prior to the second step. The second portion 120 should be properly cooled to effectuate treatment. However, special care should be given to not cool the device so as to risk irritating the ear canal 25 when in operation.

The second step of the method is to insert the enhanced earplug 100 through the outer ear 20 and into the ear canal 25. This step includes positioning the deformable bladder 130 proximate to the tympanic membrane 26 to ensure the now refrigerated coolant gel 140 interacts with the vestibular system 50, including the various otolith organs. Special care should be given to prevent having the deformable bladder 130 from actually touching, rubbing or contacting the tympanic membrane 26.

The third step of the method is to maintain the second portion 140 into the ear canal 25 in order to cool the various components of the vestibular system 50. More specifically, the invention contemplates applying a coolant in the form of the deformable bladder 140 to cool, the endolymphatic fluid in the semi-circular canals as well as the vestibular nerves and the octoconia crystals within the otolith organs. By cooling these various components of the vestibular system 50, there is a decreased sensitivity when the various otoconia crystals interact and defect ciliary bundles of hair cells within the semi-circular canals and otolith organs. This decreased sensitivity reduces the risk of confusion with visually perceived motion and accordingly reduces symptoms associated with motion sickness.

It is preferable to maintain the second portion 130 within the ear canal 25 between 10 to 30 minutes per treatment. After this amount of time, the enhanced earplug 100 should be removed for the ear canal through use of the handle 112 located on the first portion 110.

The invention is also directed towards a regiment of performing the four above referenced steps (refrigeration, insertion, cooling and removal) for a prescribed period of time prior to engaging in an activity which risks the onset of motion sickness. Such activities include riding in a car or airplane, or perhaps taking a leisure cruise on a commercial vessel.

There are two preferred regiments contemplated by the invention: a pre-activity treatment and a continuing activity treatment. The pre-activity treatment teaches application of the four-step program articulated above for a three-day stretch prior to engaging in the motion related activity. This includes a single treatment per day for a period of approximately 10 to 30 minutes—prior to an event such as airplane travel or prior to boarding a cruise.

By performing these three pre-activity treatments—the result is an adaptation. More specifically, by repeatedly cooling the vestibular system 50 there is a training of the semi-circular canals and otolith organs to become less sensitive and less susceptible to confusion caused by visually perceived movement. Accordingly, when the enhanced earplug 100 is not within the ear canal 25, there is nonetheless a decreased sensitivity and accordingly less risk of symptoms associated with motion sickness.

To maintain this adaptation, the invention contemplates a continuing activity treatment through performance of the four-step method articulated above. Accordingly, for each day of a continued activity, which risks motion sickness, the user should perform the four-step method each of those days for a period of 10 to 30 minutes. This will continue to train and essentially program the vestibular system 50 to become less sensitive and accordingly accord less confusing signals to the brain in comparison with visually perceived movement. Such adaptation should occur for at least a 24-hour period and extend up and onto the next treatment.

1. A motion sickness relief device, comprising:
a first portion having a handle and a connector;
a second portion having a first end and a corresponding second end wherein the first end is affixed to the first portion through the connector, the second portion also including a deformable bladder having a cavity; and
a quantity of coolant maintained within the cavity of the deformable bladder.

2. The motion sickness treatment device of claim 1, further comprising:
one or more diaphragms capable of creating a seal between the first portion and an ear canal opening.

3. The motion sickness treatment device of claim 2, further comprising:
an engagement rod capable of maintaining and securing the handle, the one or more diaphragms and the connector of the first portion.

4. The motion sickness treatment device of claim 1, wherein:
the deformable bladder includes a cylindrical sheath.

5. The motion sickness treatment device of claim 1, wherein:
The quantity of coolant is positioned within the cavity of the deformable bladder in order cool a user's vestibular system.

6. A method of relieving motion sickness, the method comprising the steps of:
(a) refrigerating an enhanced earplug, the enhanced earplug having:
a first portion having a handle and connector,
a second portion having a first end and a corresponding second end where the first end is affixed to the first portion through the connector, the second portion also including a deformable bladder having a cavity, and
a quantity of coolant maintained within the cavity of the deformable bladder;
(b) inserting the enhanced earplug into a user's ear canal through use of the handle;
(c) cooling the vestibular system of the user through a proximity of the coolant positioned within the deformable bladder; and
(d) removing the enhanced earplug from the ear canal after a prescribed period of time; and
(e) repeating steps (a)-(d) as necessary prior to engaging in an event which could cause symptoms associated with motion sickness.

7. The method of claim 6, further comprising the step of:
repeating steps (a)-(d) each day during a prolonged event that could cause the symptoms associated with motion sickness.

8. The method of claim 6, further comprising:
creating a seal between the first portion and an ear canal opening using one or more diaphragms.

9. The method of claim 8, further comprising:
maintaining and securing the handle, the one or more diaphragms and the connector of the first portion using an engagement rod.

10. The method of claim 6, wherein:
the deformable bladder includes a cylindrical sheath.

11. The method of claim 6, wherein:
a sufficient quantity of coolant is positioned within the cavity of the deformable bladder in order cool a user's vestibular system.
